(19) United States
(12) Patent Application Publication
(10) Pub. No.: US 2025/0387307 A1
(43) Pub. Date: Dec. 25, 2025

(54) PRODUCTION METHOD FOR A SOLID PARTICLE

(71) Applicant: KAO CORPORATION, Tokyo (JP)

(72) Inventors: Saki HAMADA, Chuo-ku (JP); Hayato MIYAZAKI, Chuo-ku (JP); Shinji OGURI, Chuo-ku (JP)

(73) Assignee: KAO CORPORATION, Tokyo (JP)

(21) Appl. No.: 18/752,054

(22) Filed: Jun. 24, 2024

(51) Int. Cl. A61K 8/11; A61K 8/25

(52) U.S. Cl. CPC A61K 8/11; A61K 8/25; A61K 2800/621

(57) ABSTRACT

Provided is a production method for a solid particle, including: heating a raw material composition containing an oily component to impart fluidity to the raw material composition; granulating the raw material composition having imparted thereto the fluidity through ejection to form a granular raw material; and dropping the granular raw material into powder to coat a surface of the granular raw material with the powder, wherein, at a time of the formation of the granular raw material, a maximum length of elongation of the raw material composition at a time of the granulation of the raw material composition through the ejection is measured in advance, and a length of a dropping distance of the granular raw material is set so that a ratio of the length of the dropping distance to the maximum length of the elongation at the time of the granulation is more than 0 and 9 or less.

### TECHNICAL FIELD

The present invention relates to a production method for a solid particle.

### BACKGROUND ART

Solid particles that have heretofore been known such as solid cosmetics have been generally in such a form that powder is subjected to compression molding to be stored in a shallow-bottom tray like a foundation or in such a form that a composition that is a solid at room temperature is molded into a predetermined shape like a lipstick. In addition to the solid cosmetics in such forms, a solid cosmetic in a granular form (granular solid cosmetic) has been proposed in recent years.

In, for example, Japanese Patent Application Laid-open No. 2021-109850 (Patent Literature 1), there is a description of a production method for a granular solid cosmetic including granulating a raw material for a cosmetic and causing powder to adhere to the surface of the granulated raw material for a cosmetic.

### SUMMARY OF THE INVENTION

The present invention relates to a production method for a solid particle including: heating a raw material composition containing an oily component to impart fluidity to the raw material composition; granulating the raw material composition having imparted thereto the fluidity through ejection to form a granular raw material; and dropping the granular raw material into powder to coat a surface of the granular raw material with the powder,
wherein (i) at a time of the formation of the granular raw material, a maximum length of elongation of the raw material composition at a time of the granulation of the raw material composition through the ejection is measured in advance, and a length of a dropping distance of the granular raw material is set so that a ratio of the length of the dropping distance to the maximum length of the elongation at the time of the granulation is more than 0 and 9 or less, or
wherein (ii) at a time of the coating of the surface of the granular raw material with the powder, an airflow is applied from a side surface of a dropping track of the granular raw material.

The granular solid cosmetic described in Patent Literature 1 has involved a problem in that, at a time of the granulation of the raw material for a cosmetic, a fine particle is formed in addition to a desired granulated cosmetic, and the fine particle adheres to the granular solid cosmetic.

The present invention relates to a production method for a solid particle in which the adhesion of a fine particle is suppressed.

The inventors of the present invention have found that, at a time of dropping of a granular raw material formed by granulating a raw material composition containing an oily component having imparted thereto fluidity by heating into powder to coat the surface of the granular raw material with the powder, (i) when the raw material composition is dropped from an ejection orifice of a nozzle at a time of formation of the granular raw material, a solid particle to which no fine particle adheres is obtained in a high yield by determining the length of a dropping distance with respect to the maximum length of elongation of the raw material composition from the ejection orifice, or (ii) at a time of the coating of the surface of the granular raw material with the powder, a solid particle to which no fine particle adheres is obtained in a high yield by applying an airflow from the side surface of a dropping track.

The present invention relates to the following items [1] and [2].

[1] A production method for a solid particle, including: heating a raw material composition containing an oily component to impart fluidity to the raw material composition; granulating the raw material composition having imparted thereto the fluidity through ejection to form a granular raw material; and dropping the granular raw material into powder to coat a surface of the granular raw material with the powder, wherein, at a time of the formation of the granular raw material, a maximum length of elongation of the raw material composition at a time of the granulation of the raw material composition through the ejection is measured in advance, and a length of a dropping distance of the granular raw material is set so that a ratio of the length of the dropping distance to the maximum length of the elongation of the raw material composition at the time of the granulation is more than 0 and 9 or less.

[2] A production method for a solid particle, including: heating a raw material composition containing an oily component to impart fluidity to the raw material composition; granulating the raw material composition having imparted thereto the fluidity through ejection to form a granular raw material; and dropping the granular raw material into powder to coat a surface of the granular raw material with the powder, wherein at a time of the coating of the surface of the granular raw material with the powder, an airflow is applied from a side surface of a dropping track of the granular raw material.

### BRIEF DESCRIPTION OF DRAWINGS

FIG. 1 is a schematic view for illustrating a mechanism through which fine particles adhere to solid particles.

FIG. 2 is a schematic view for illustrating a first aspect of a production method according to at least one embodiment of the present invention.

FIG. 3 is a schematic view for illustrating the first aspect of the production method according to at least one embodiment of the present invention including using a vibration feeder.

FIG. 4 is a schematic view for illustrating a second aspect of the production method according to at least one embodiment of the present invention.

FIG. 5 is a schematic view for illustrating the second aspect of the production method according to at least one embodiment of the present invention including using a vibration feeder.

FIG. 6 is a schematic view for illustrating solid particles to which no fine particles adhere.

FIG. 7 is a schematic view for illustrating solid particles to which fine particles adhere.

### DETAILED DESCRIPTION OF THE INVENTION

The present invention is described below on the basis of its exemplary embodiments with reference to the drawings. The present invention relates to a production method for a solid particle.

### [Production Method for Solid Particle]

A first aspect of a production method for a solid particle according to at least one embodiment of the present invention includes: heating a raw material composition containing an oily component to impart fluidity to the raw material composition; granulating the raw material composition having imparted thereto the fluidity through ejection to form a granular raw material; and dropping the granular raw material into powder to coat a surface of the granular raw material with the powder.

At the time of the formation of the granular raw material, the maximum length of elongation of the raw material composition at the time of the granulation of the raw material composition through the ejection is measured in advance, and a length of a dropping distance of the granular raw material is set so that a ratio of the length of the dropping distance to the maximum length of the elongation at the time of the granulation is more than 0 and 9 or less.

In addition, a second aspect of a production method for a solid particle according to at least one embodiment of the present invention includes: heating a raw material composition containing an oily component to impart fluidity to the raw material composition; granulating the raw material composition having imparted thereto the fluidity through ejection to form a granular raw material; and dropping the granular raw material into powder to coat a surface of the granular raw material with the powder.

At the time of the coating of the surface of the granular raw material with the powder, an airflow is applied from a side surface of a dropping track of the granular raw material.

As used herein, the term “raw material” in each of the terms “raw material composition” and “granular raw material” means a raw material for a solid particle that is a product and does not limit a material or the like.

The production method for a solid particle according to at least one embodiment of the present invention exhibits such an effect that a solid particle to which no fine particle adheres is obtained in a high yield. The reason why the production method exhibits such effect is not clear but is conceived to be as described below.

The inventors of the present invention have found that, when a granular raw material formed in a granular shape by ejecting a raw material composition containing an oily component having imparted thereto fluidity by heating is dropped into powder, the raw material composition having imparted thereto the fluidity is elongated to form a liquid column, and when the granular raw material is cut to be separated from the liquid column, a fine particle is formed secondarily in addition to the granular raw material. It is conceived that the fine particle falls in the same track as that of the granular raw material to adhere to the granular raw material at the time of the dropping into the powder, and thus a solid particle to which a fine particle adheres (hereinafter also referred to as “fine particle-adhered solid particle”) is generated.

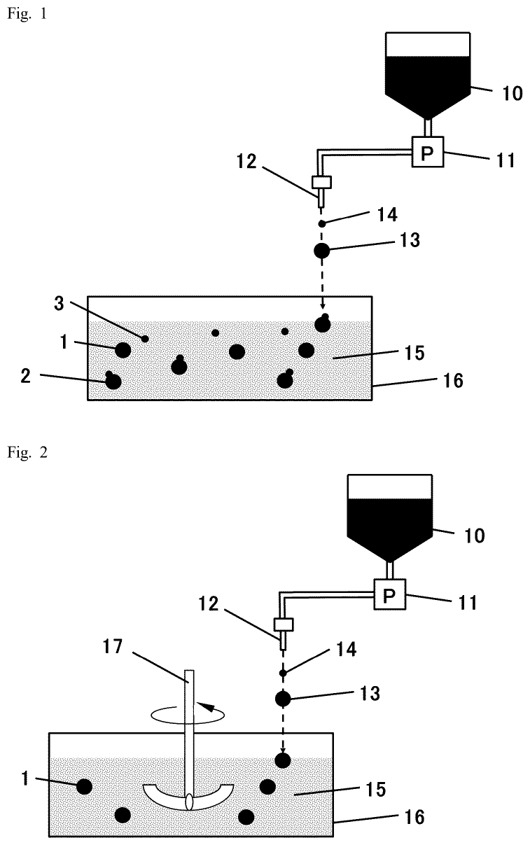

The mechanism through which the fine particle-adhered solid particle is generated, and the effects of the present invention are described in detail below with reference to FIG. 1. When a raw material composition 10 having imparted thereto fluidity by heating the raw material composition is delivered with a pump 11 or the like, and is ejected to be dropped from the tip of a nozzle 12. At this time, the raw material composition is elongated and cut to be granulated to form a granular raw material 13 and a fine particle 14. The falling tracks of the granular raw material 13 and the fine particle 14 are the same, but the fine particle 14 falls later than the granular raw material does. The inventors of the present invention have found the following. When the granular raw material 13 is dropped into powder 15, the granular raw material 13 is rapidly cooled. When the fine particle 14 that has fallen later lands at a position slightly shifted from the center in the vertical direction of the cooled granular raw material 13, a fine particle-adhered solid particle 2 in a state in which the fine particle 14 adheres to the granular raw material 13 without coalescing therewith is obtained. When the fine particle 14 lands at substantially the same position as the center in the vertical direction of the granular raw material 13, the fine particle 14 coalesces with the granular raw material 13 to provide one granular raw material 13. In addition, when the fine particle 14 falls to a position shifted by a distance exceeding the radius from the center of the granular raw material 13, the surfaces of the dropped granular raw material 13 and fine particle 14 are each coated with the powder 15 (the granular raw material 13 and the fine particle 14 each coated with the powder are hereinafter referred to as “solid particle 1” and “satellite particle 3,” respectively), and hence both the particles do not adhere to each other. The inventors of the present invention have also found that, when the dropping distance from the tip of the nozzle 12 to the surface of the powder 15 is somewhat short, the fine particle 14 is not generated as a by-product.

In addition, when the raw material composition 10 having imparted thereto fluidity by heating the raw material composition is delivered with the pump 11 or the like, and is ejected to be dropped from the tip of the nozzle 12. At this time, the raw material composition is elongated and cut to be granulated to form the granular raw material 13 and the fine particle 14. The granular raw material 13 and the fine particle 14 become the solid particle 1 and the satellite particle 3, respectively, when the surfaces thereof are each coated with the powder 15.

Meanwhile, the inventors of the present invention have found the following. The falling tracks of the granular raw material 13 and the fine particle 14 are the same, and the fine particle 14 falls later than the granular raw material 13 does. Thus, when the fine particle 14 that has fallen later lands on the granular raw material 13 cooled by the powder 15, the fine particle-adhered solid particle 2 may be obtained.

The inventors of the present invention have made various investigations based on the above-mentioned findings, and as a result, have found the following: (i) when the maximum length of the elongation of the raw material composition at the time of the granulation through the ejection is measured in advance, and the length of a dropping distance of the granular raw material is set so that the ratio of the length to the maximum length of the elongation at the time of the granulation falls within a specific range, the generation of the fine particle 14 as a by-product as described above does not occur, or even if the fine particle 14 is generated as a by-product, the fine particle 14 lands on the granular raw material 13 directly in front thereof in the cooling of the vertical direction before the granular raw material 13, resulting in the coalescence of the granular raw material 13 and the fine particle 14, and hence a spherical solid particle to which no fine particle adheres is obtained in a high yield; and (ii) the dropping positions of the granular raw material and the fine particle into the powder may be set to different positions by applying an airflow from the side surface of the dropping track at the time of the dropping of the granular raw material into the powder and utilizing a difference in magnitude of the ratio of the mass of the particle to the front projected area in the direction of the airflow, and hence the solid particle 1 to which no fine particle adheres is obtained in a high yield.

The term “fine particle” as used herein means a particle to be generated secondarily at the time of the formation of the granular raw material, the particle having a particle diameter smaller than that of the granular raw material. Specifically, the fine particle is a particle having a diameter smaller than one-third of the diameter of the granular raw material.

In addition, the term “coalesce” in, for example, the coalesce of the granular raw material 13 and the fine particle 14 as used herein means that the fine particle 14 is incorporated into the granular raw material 13 to become indistinguishable in appearance therefrom. The term “adhesion” in, for example, the adhesion of the fine particle 14 to the granular raw material 13 as used herein means that the fine particle 14 and the granular raw material 13 form one particle under a state in which a portion derived from the fine particle 14 and a portion derived from the granular raw material 13 are distinguishable in appearance from each other.

### [First Aspect]

An example of the first aspect of the production method for a solid particle according to at least one embodiment of the present invention is described below with reference to FIG. 2.

In the production method according to at least one embodiment of the present invention, the expression “coat the surface of a granular raw material 13 with powder 15” means not only that the entirety of the surface of the granular raw material 13 is coated with the powder 15 but also that at least part of the surface of the granular raw material 13 is coated with the powder 15. From the viewpoint of improving the adhesion resistance and transportation resistance of the solid particle, the entirety of the surface of the granular raw material 13 is preferably coated with the powder 15.

### [Impartment of Fluidity to Raw Material Composition]

The impartment of fluidity to the raw material composition is performed by heating the raw material composition containing the oily component. The raw material composition 10 having imparted thereto the fluidity may be obtained by heating the raw material composition to a temperature equal to or more than the melting point of at least one substance in the raw material composition. In addition, the heating preferably includes heating the raw material composition to its melting point or more.

A temperature for imparting the fluidity to the raw material composition is preferably 60° C. or more, more preferably 70° C. or more, still more preferably 80° C. or more, still more preferably 85° C. or more from the viewpoint of retarding the solidification of the granular raw material 13 to accelerate the adhesion of the powder 15 at the time of the coating of the surface of the granular raw material 13 with the powder 15. In addition, the temperature is preferably 150° C. or less, more preferably 130° C. or less, still more preferably 120° C. or less, still more preferably 115° C. or less from the viewpoint of preventing the deterioration of the raw material composition due to heat. In particular, the temperature for imparting the fluidity to the raw material composition is preferably 60° C. or more and 150° C. or less, more preferably 70° C. or more and 130° C. or less, still more preferably 80° C. or more and 120° C. or less, still more preferably 85° C. or more and 115° C. or less.

### [Formation of Granular Raw Material]

In the formation of the granular raw material, the raw material composition 10 having imparted thereto the fluidity is granulated to form the granular raw material 13. When the raw material composition 10 having imparted thereto the fluidity illustrated in FIG. 2 is a liquid obtained by heating the raw material composition to its melting point or more, the ejection of the raw material composition 10 delivered with the pump 11 from the tip of the nozzle 12 and the elongation and cutting of the raw material composition 10 can form the granular raw material 13 as a droplet. In addition, an apparatus for forming the granular raw material 13 from the raw material composition 10 having imparted thereto the fluidity is not limited to an apparatus illustrated in FIG. 2, and a known droplet-producing apparatus or the like may be used.

Regarding the raw material composition 10 having imparted thereto the fluidity, the maximum length of the elongation of the raw material composition 10 at the time of the granulation through the ejection is measured in advance. The maximum length of the elongation is influenced not only by the raw material composition 10 but also by conditions, such as an ejection temperature and a flow rate, in the formation of the granular raw material, and is specific to individual production conditions. Thus, the maximum length of the elongation needs to be measured before the present invention is carried out. In addition, in the formation of the granular raw material, in the case where the length of the dropping distance of the granular raw material 13 is set so that its ratio to the maximum length of the elongation of the raw material composition 10 at the time of the granulation is more than 0 and 9 or less, when the ratio is more than 0 and less than 1, the fine particle 14 is less liable to be formed though the formation depends on the viscosity and the like of the raw material composition 10, and when the ratio is 1 or more and 9 or less, the fine particle 14 is formed, but the granular raw material 13 and the fine particle 14 coalesce with each other at the time of the dropping of the granular raw material 13 into the powder 15. Thus, a solid particle to which no fine particle adheres can be obtained in a high yield.

The ratio of the length of the dropping distance of the granular raw material 13 to the maximum length of the elongation of the raw material composition 10 at the time of the granulation is preferably 0.1 or more, more preferably 0.2 or more from the viewpoint of stably obtaining the granular raw material 13 having a uniform size, and is preferably 8.5 or less, more preferably 8 or less, still more preferably 6 or less, still further more preferably 5.5 or less from the viewpoint of making the generation of the fine particle 14 as a by-product difficult or causing the fine particle 14 that has been generated as a by-product to coalesce with the granular raw material 13 to provide the solid particle 13 to which no fine particle 14 adheres.

The distance at which the granular raw material 13 is dropped, that is, the distance between the tip of the nozzle 12 and the outermost surface of the layer of the powder 15 is preferably 200 mm or less, more preferably 170 mm or less, still more preferably 150 mm or less, still further more preferably 120 mm or less, still further more preferably 90 mm or less from the viewpoints of alleviating impact at the time of the contact of the dropped granular raw material 13 with the powder 15 to prevent the deformation of the granular raw material 13 and suppressing the cooling of the granular raw material 13 during the dropping to cause the granular raw material 13 and the fine particle 14 to coalesce with each other. In addition, it is only required that the dropping distance be more than 0 mm, that is, the powder 15 and the nozzle 12 be out of contact with each other. The dropping distance is preferably 2 mm or more, more preferably 3 mm or more, still more preferably 4 mm or more from the viewpoint of forming the granular raw material 13 into a spherical shape.

The maximum length of the elongation of the raw material composition 10 at the time of the granulation is the distance between the tip of the nozzle 12 and the lower end of a droplet (hereinafter also referred to as “base particle”) formed at the tip of the elongated raw material composition 10 immediately before the elongated raw material composition 10 is cut at any point to form the granular raw material 13 when the raw material composition 10 is liquefied and ejected from the nozzle 12 at a sufficiently long dropping distance. The distance at which the granular raw material 13 is dropped in the present invention depends on the maximum length of the elongation of the raw material composition 10 as described above, and the length is influenced by the outer diameter of the nozzle 12, the composition and temperature of the raw material composition 10, and the flow rate of the raw material composition 10 at the time of the ejection from the nozzle 12. Thus, it is required that the dropping distance be set again every time the conditions for producing the granular raw material 13 are changed. The timing of the “immediately before” is determined by a method described in Examples.

The maximum length of the elongation of the raw material composition 10 at the time of the granulation is preferably 25 mm or less, more preferably 20 mm or less, still more preferably 18 mm or less from the viewpoint of causing the granular raw material and the fine particle to coalesce with each other to provide a solid particle to which no fine particle adheres. There is no limitation on the lower limit because the issue of the present invention does not arise unless the raw material composition 10 is elongated at the time of the granulation. In general, when the length of the elongation of the raw material composition 10 at the time of the granulation is 2 mm or more, a solid particle to which a fine particle adheres may be generated.

The maximum length of the elongation of the raw material composition 10 at the time of the granulation may be measured by a method described in Examples.

The solid particle 1 that is the object of the production method according to at least one embodiment of the present invention includes the core portion and the shell portion formed of the layer of the raw material composition that has incorporated therein the powder for coating the core portion, and hence the size thereof is substantially the same as or slightly larger than that of the granular raw material 13 formed by the elongation and cutting of the raw material composition 10. That is, the granular raw material 13 is preferably adjusted based on the average projected area, diameter, and/or weight of the solid particle 1 to be obtained when being placed on a plane.

The particle diameter of the granular raw material 13 mainly correlates with the outer diameter of the nozzle. When the outer diameter of the nozzle is increased, the particle diameter of the granular raw material 13 is increased. To this end, for example, when the granular raw material 13 is formed as a droplet by ejecting the raw material composition 10 that is a liquid from the tip of the nozzle 12, the outer diameter of the nozzle 12 may be changed in accordance with the particle diameter of the target solid particle. The outer diameter of the nozzle 12 is preferably 0.5 mm or more, more preferably 1 mm or more, still more preferably 1.5 mm or more from the viewpoint of obtaining a particle diameter corresponding to the usage amount of the solid particle 1 per time. In addition, the outer diameter is preferably 20 mm or less, more preferably 10 mm or less, still more preferably 5 mm or less from the viewpoint of stably dropping the granular raw material 13. In particular, the outer diameter of the nozzle 12 is preferably 0.5 mm or more and 20 mm or less, more preferably 1 mm or more and 10 mm or less, still more preferably 1.5 mm or more and 5 mm or less.

[Coating of Surface of Granular Raw Material with Powder]

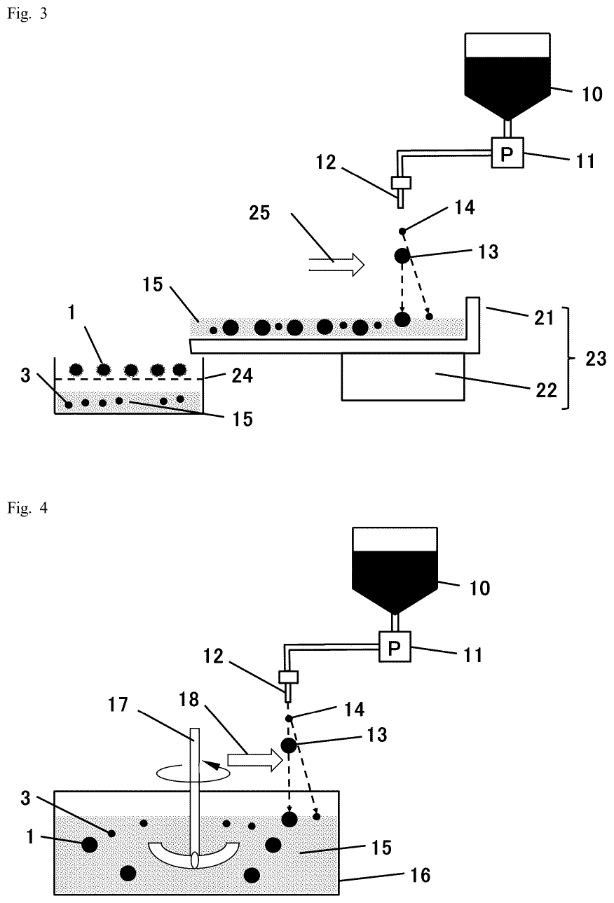

In the coating of the surface of the granular raw material with the powder, the granular raw material 13 is dropped into the powder 15 to cause the granular raw material 13 and the fine particle 14 to coalesce with each other as required to coat the surface of the granular raw material 13 with the powder 15, to thereby provide the solid particle 1. The powder 15 may be stored in a container 16. In addition, when such an apparatus as illustrated in FIG. 3 to be described later is used, the powder 15 falls to a sieve 24 through the operation of a vibration feeder 23, and may be continuously supplied with a powder-supplying device in accordance with the falling speed so that the amount of the powder 15 on a trough 21 was constant.

The “granular raw material 13” at the time of the coating of the surface of the granular raw material with the powder encompasses the granular raw material 13 and the fine particle 14 that have coalesced with each other.

The thickness of the layer of the powder 15 is preferably 50 mm or more, more preferably 60 mm or more, still more preferably 70 mm or more from the viewpoint of accelerating the adhesion of the powder 15 to the upper portion of the granular raw material 13 that has been dropped, and the thickness is preferably 250 mm or less, more preferably 230 mm or less, still more preferably 210 mm or less from the viewpoint of avoiding excessive use of the powder. In addition, when the apparatus as illustrated in FIG. 3 to be described later is used, the thickness of the layer of the powder 15 is preferably 3 mm or more, more preferably 5 mm or more, still more preferably 8 mm or more from the viewpoint of accelerating the adhesion of the powder 15 to the upper portion of the granular raw material 13 that has been dropped. In addition, the thickness of the layer of the powder 15 is preferably 50 mm or less, more preferably 30 mm or less, still more preferably 20 mm or less, still more preferably 15 mm or less from the viewpoint of avoiding excessive use of the powder.

The temperature of the powder 15 is preferably 5° C. or more, more preferably 15° C. or more, still more preferably 20° C. or more from the viewpoint of retarding the solidification of the granular raw material 13 to accelerate the adhesion of the powder 15. In addition, the temperature of the powder 15 is preferably 60° C. or less, more preferably 55° C. or less, still more preferably 50° C. or less, still further more preferably 30° C. or less from the viewpoint of suppressing excessive adhesion of the powder 15 to the granular raw material 13. In particular, the temperature of the powder 15 is preferably 5° C. or more and 60° C. or less, more preferably 15° C. or more and 55° C. or less, still more preferably 20° C. or more and 50° C. or less, still further more preferably from 20° C. to 30° C. (normal temperature).

The time period for which the surface of the granular raw material 13 is coated with the powder 15 is preferably 2 seconds or more, more preferably 3 seconds or more, still more preferably 4 seconds or more from the viewpoint of accelerating the adhesion of the powder to the surface of the granular raw material 13. In addition, the time period for which the surface of the granular raw material 13 is coated with the powder 15 is preferably 24 hours or less, more preferably 12 hours or less, still more preferably 6 hours or less from the viewpoint of productivity.

When the powder 15 is stirred with a stirrer 17, the granular raw material 13 dropped into the powder 15 moves from the dropping position before the next granular raw material 13 is dropped, and hence the granular raw materials 13 can be prevented from adhering to each other.

The coating of the surface of the granular raw material 13 with the powder 15 may be performed under a state in which vibration is applied to the powder 15. For example, in FIG. 3, the use of the vibration feeder 23 in which the trough 21 is arranged on a vibration device 22 applies the vibration to the powder 15 on the trough 21. When the granular raw material 13 is dropped into the powder 15 having applied thereto the vibration, the surface of the granular raw material 13 can be coated with the powder 15 under a state in which the vibration is applied to the powder 15. At this time, it is preferred that the powder 15 be continuously supplied onto the trough 21 with the powder-supplying device (not shown).

The granular raw material 13 and the powder 15 are brought into contact with each other under a state in which the vibration is applied to the powder 15 to cause the powder 15 to adhere to the granular raw material 13, to thereby coat the surface of the granular raw material 13 with the powder 15. Thus, the powder 15 can be incorporated to a depth of at least about 80 μm from the surface of the granular raw material 13, and hence a solid particle excellent in adhesion resistance and transportation resistance is obtained.

In addition, a bowl may be used instead of the trough 21 as a container for storing the powder 15. Through use of a bowl feeder in which the bowl is arranged on the vibration device, the granular raw material that has been brought into contact with the powder having applied thereto the vibration ascends a slope arranged on the inner wall of the bowl through the vibration while the surface thereof is coated with the powder. To lengthen the time period for which the granular raw material and the powder are brought into contact with each other, when the trough 21 is used, the trough needs to be lengthened. However, when the bowl is used, space efficiency is satisfactory because an increase in number of spiral turns of the slope in the height direction thereof suffices for the purpose.

The amplitude of the vibration to be applied to the powder 15 at the time of the coating of the surface of the granular raw material 13 with the powder 15 is preferably 0.3 mm or more, more preferably 0.4 mm or more, still more preferably 0.5 mm or more, still more preferably 0.6 mm or more from the viewpoint of accelerating the adhesion of the powder 15 on the surface of the granular raw material 13. In addition, the amplitude of the vibration is preferably 5 mm or less, more preferably 4 mm or less, still more preferably 3 mm or less, still further more preferably 1.5 mm or less, still further more preferably 1.4 mm or less, still further more preferably 1.3 mm or less. In particular, the amplitude of the vibration is preferably 0.3 mm or more and 5 mm or less, more preferably 0.4 mm or more and 4 mm or less, still more preferably 0.5 mm or more and 4 mm or less, still more preferably 0.5 mm or more and 3 mm or less, still further more preferably 0.6 mm or more and 3 mm or less, still further more preferably 0.3 mm or more and 1.5 mm or less, still further more preferably 0.4 mm or more and 1.4 mm or less, still further more preferably 0.5 mm or more and 1.3 mm or less, still further more preferably 0.6 mm or more and 1.3 mm or less.

The amplitude of the vibration to be applied to the powder 15 is preferably measured at a position directly above the vibration device 22.

In addition, the frequency of the vibration is preferably 30 Hz or more, more preferably 40 Hz or more, still more preferably 50 Hz or more from the viewpoint of accelerating the adhesion of the powder 15 to the surface of the granular raw material 13. In addition, the frequency of the vibration is preferably 300 Hz or less, more preferably 100 Hz or less, still more preferably 75 Hz or less, still further more preferably 60 Hz or less. In particular, the frequency of the vibration is preferably 30 Hz or more and 300 Hz or less, more preferably 40 Hz or more and 100 Hz or less, still more preferably 50 Hz or more and 75 Hz or less, still further more preferably 50 Hz or more and 60 Hz or less.

The time period for which the surface of the granular raw material 13 is coated with the powder 15 in the apparatus illustrated in FIG. 3 is preferably 2 seconds or more, more preferably 3 seconds or more, still more preferably 4 seconds or more from the viewpoint of accelerating the adhesion of the powder to the surface of the granular raw material 13. In addition, the time period for which the surface of the granular raw material 13 is coated with the powder 15 is preferably 300 seconds or less, more preferably 200 seconds or less, still more preferably 100 seconds or less from the viewpoint of productivity. In particular, the time period for which the surface of the granular raw material 13 is coated with the powder 15 is preferably 2 seconds or more and 300 seconds or less, more preferably 3 seconds or more and 200 seconds or less, still more preferably 4 seconds or more and 100 seconds or less.

Through the above-mentioned coating of the surface of the granular raw material with the powder, the powder 15 is caused to adhere to the surface of the granular raw material 13, and the surface of the granular raw material 13 is coated with the powder 15. Thus, the solid particle 1 can be obtained.

### [Second Aspect]

An example of the second aspect of the production method for a solid particle according to at least one embodiment of the present invention is described below with reference to FIG. 4.

### [Impartment of Fluidity to Raw Material Composition]

The impartment of fluidity to the raw material composition is performed by heating the raw material composition containing the oily component to impart the fluidity thereto, and is the same as the impartment of the fluidity to the raw material composition in the above-mentioned first aspect.

### [Formation of Granular Raw Material]

In the formation of the granular raw material, the raw material composition 10 having imparted thereto the fluidity is granulated to form the granular raw material 13. The raw material composition 10 having imparted thereto the fluidity illustrated in FIG. 4 is delivered with the pump 11, and is granulated through ejection from the tip of the nozzle 12 to form the granular raw material 13. When the raw material composition 10 is a liquid obtained by heating the raw material composition to its melting point or more, the ejection of the raw material composition 10 delivered by the pump 11 from the tip of the nozzle 12 can form the granular raw material 13 as a droplet. In addition, an apparatus for forming the granular raw material 13 from the raw material composition 10 having imparted thereto the fluidity is not limited to an apparatus illustrated in FIG. 4, and a known droplet-producing apparatus or the like may be used.

The granulation method is not limited to the case of ejection and dropping from the nozzle 12 as illustrated in FIG. 4, and can also be performed by methods involving changing the ejection direction from the nozzle, cutting the flowed raw material composition 10 with a shutter, and the like.

The solid particle 1 that is the object of the production method according to at least one embodiment of the present invention includes the core portion and the shell portion formed of the layer of the raw material composition that has incorporated therein the powder for coating the core portion, and hence the size thereof is substantially the same as or slightly larger than that of the granular raw material 13 formed by the elongation and cutting of the raw material composition 10. That is, the granular raw material 13 is preferably adjusted based on the average projected area, diameter, and/or weight of the solid particle 1 to be obtained when being placed on a plane.

The particle diameter of the granular raw material 13 mainly correlates with the outer diameter of the nozzle. When the outer diameter of the nozzle is increased, the particle diameter of the granular raw material 13 is increased. To this end, for example, when the granular raw material 13 is formed as a droplet by ejecting the raw material composition 10 that is a liquid from the tip of the nozzle 12, the outer diameter of the nozzle 12 may be changed in accordance with the particle diameter of the target solid particle 1. The outer diameter of the nozzle 12 is the same as that of the nozzle 12 described in the formation of the granular raw material in the above-mentioned first aspect.

[Coating of Surface of Granular Raw Material with Powder]

In the coating of the surface of the granular raw material with the powder, the granular raw material 13 is dropped into the powder 15 to coat the surface of the granular raw material 13 with the powder 15, to thereby provide the solid particle 1. The powder 15 may be stored in the container 16. At this time, the fine particle 14 formed secondarily at the time of the formation of the granular raw material 13 is moved in a horizontal direction through the application of an airflow 18 from the side surface of the dropping track of the granular raw material 13, and thus the dropping positions of the granular raw material 13 and the fine particle 14 to the powder 15, that is, the position at which the granular raw material 13 is first brought into contact with the powder 15 and the position at which the fine particle 14 is first brought into contact with the powder 15 are set to different positions.

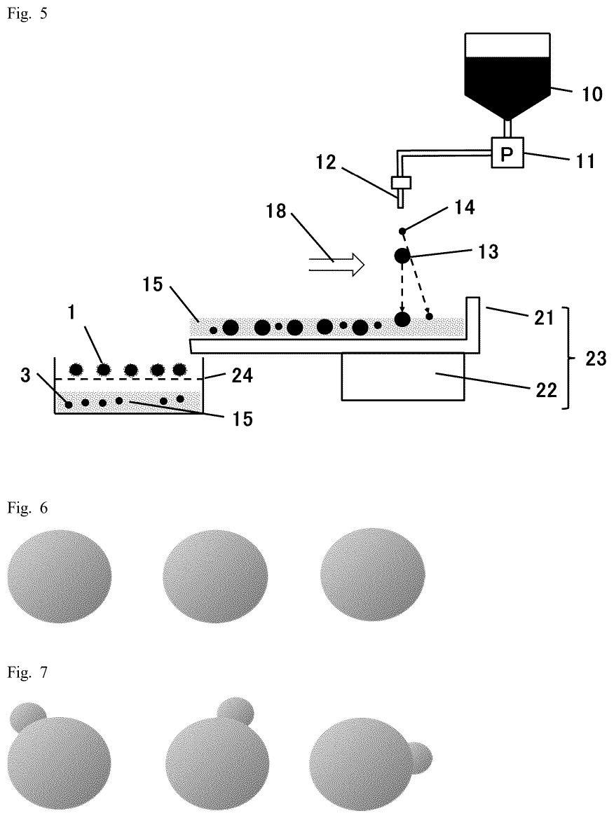

In addition, when such an apparatus as illustrated in FIG. 5 to be described later is used, the powder 15 falls to the sieve 24 through the operation of the vibration feeder 23, and may be continuously supplied with a powder-supplying device in accordance with the falling speed so that the amount of the powder 15 on the trough 21 was constant.

The distance at which the granular raw material 13 is dropped, that is, the distance between the tip of the nozzle 12 and the outermost surface of the layer of the powder 15 is preferably 350 mm or less, more preferably 250 mm or less, still more preferably 150 mm or less from the viewpoints of alleviating impact at the time of the contact of the dropped granular raw material 13 with the powder 15 to prevent the deformation of the granular raw material 13 and suppressing the cooling of the granular raw material 13 during the dropping, and the distance is preferably 50 mm or more, more preferably 70 mm or more, still more preferably 90 mm or more from the viewpoint of setting the dropping positions of the granular raw material 13 and the fine particle 14 to the powder to different positions.

The thickness of the layer of the powder 15 in the case of using the apparatus illustrated in FIG. 4 is the same as the thickness of the layer of the powder 15 at the time of the coating of the surface of the granular raw material with the powder described in the case of using the apparatus illustrated in FIG. 2 in the above-mentioned first aspect, and the thickness of the layer of the powder 15 in the case of using the apparatus illustrated in FIG. 5 is the same as the thickness of the layer of the powder 15 at the time of the coating of the surface of the granular raw material with the powder described in the case of using the apparatus illustrated in FIG. 3 in the above-mentioned first aspect.

The temperature of the powder 15 is the same as the temperature of the powder 15 at the time of the coating of the surface of the granular raw material with the powder in the above-mentioned first aspect.

The time period for which the surface of the granular raw material 13 is coated with the powder 15 is the same as the time period for which the surface of the granular raw material 13 is coated with the powder 15 at the time of the coating of the surface of the granular raw material with the powder in the above-mentioned first aspect.

At the time of the coating of the surface of the granular raw material with the powder, the airflow 18 is applied from the side surface of the dropping track of the granular raw material 13 while the granular raw material 13 is dropped into the powder 15.

The strength of the airflow 18 may be any strength as long as the dropping positions of the granular raw material 13 and the fine particle 14 to the powder can be set to different positions, and the strength may be appropriately determined as required. For example, the strength of the airflow 18 may be set to the strength to such a degree that the fine particle 14 is blown out of the container 16 for storing the powder 15.

In addition, the direction in which the airflow 18 is applied may be any direction as long as the dropping positions of the granular raw material 13 and the fine particle 14 to the powder can be set to different positions. When the fine particle 14 is dropped onto the trough 21 onto which the granular raw material 13 is dropped, it is preferred that the airflow 18 be applied from a direction orthogonal to the dropping track of the granular raw material 13 from the viewpoint of simplicity. In addition, for example, when the position at which the fine particle 14 is dropped is set to be farther from the position of the granular raw material 13 without any change of the strength of the airflow 18 and when the fine particle 14 is dropped into a container arranged outside of the trough 21 instead of being dropped onto the trough 21, it is preferred that the airflow 18 be applied from a diagonally downward direction to a diagonally upward direction. The position at which the airflow 18 is applied may be any position between the position at which the granular raw material 13 is formed and the dropping position as long as the dropping positions of the granular raw material 13 and the fine particle 14 can be set to different positions, and the position may be appropriately determined as required. However, the position at which the airflow 18 is applied is preferably set to an upper portion of the dropping track in which the dropping speed is slower from the viewpoint of changing the dropping position of the fine particle 14 more efficiently.

The surfaces of the granular raw material 13 and the fine particle 14 that have been separately dropped into the powder 15 by applying the airflow 18 are each coated with the powder 15 to provide the solid particle 1 and the fine particle in which the surface is coated with the powder (hereinafter referred to as “satellite particle 3”), and hence the solid particle 1 and the satellite particle 3 rarely adhere to each other.

In addition, when the powder 15 is stirred with a stirrer 18, the granular raw material 13 dropped into the powder 15 moves from the dropping position before the next granular raw material 13 is dropped, and the granular raw materials 13 can be prevented from adhering to each other.

The coating of the surface of the granular raw material 13 with the powder 15 may be performed under a state in which vibration is applied to the powder 15. For example, in FIG. 5, the use of the vibration feeder 23 in which the trough 21 is arranged on the vibration device 22 applies the vibration to the powder 15 on the trough 21. When the granular raw material 13 is dropped into the powder 15 having applied thereto the vibration, the surface of the granular raw material 13 can be coated with the powder 15 under a state in which the vibration is applied to the powder 15. In this case, it is preferred that the powder 15 be continuously supplied onto the trough 21 with the powder-supplying device (not shown).

The granular raw material 13 and the powder 15 are brought into contact with each other under a state in which the vibration is applied to the powder 15 to cause the powder 15 to adhere to the granular raw material 13, to thereby coat the surface of the granular raw material 13 with the powder 15. Thus, the powder 15 can be incorporated to a depth of at least about 80 μm from the surface of the granular raw material 13, and hence a solid particle excellent in adhesion resistance and transportation resistance is obtained.

In addition, a bowl may be used instead of the trough 21 as a container for storing the powder 15. Through use of a bowl feeder in which the bowl is arranged on the vibration device, the granular raw material that has been brought into contact with the powder having applied thereto the vibration ascends a slope arranged on the inner wall of the bowl through the vibration while the surface thereof is coated with the powder. To lengthen the time period for which the granular raw material and the powder are brought into contact with each other, when the trough 21 is used, the trough needs to be lengthened. However, when the bowl is used, space efficiency is satisfactory because an increase in number of spiral turns of the slope in the height direction thereof suffices for the purpose.

The amplitude of the vibration to be applied to the powder 15 at the time of the coating of the surface of the granular raw material 13 with the powder 15 is the same as the amplitude of the vibration at the time of the coating of the surface of the granular raw material with the powder in the above-mentioned first aspect.

The amplitude of the vibration to be applied to the powder 15 is preferably measured at a position directly above the vibration device 22.

In addition, the frequency of the vibration is the same as the frequency of the vibration at the time of the coating of the surface of the granular raw material with the powder in the above-mentioned first aspect.

The time period for which the surface of the granular raw material 13 is coated with the powder 15 in the apparatus illustrated in FIG. 5 is the same as the time period for which the surface of the granular raw material 13 is coated with the powder 15 in the apparatus illustrated in FIG. 3 in the above-mentioned first aspect.

Through the above-mentioned coating of the surface of the granular raw material with the powder, the powder 15 is caused to adhere to the surface of the granular raw material 13, and the surface of the granular raw material 13 is coated with the powder 15. Thus, the solid particle 1 can be obtained.

### (Cooling)

In each of the first aspect and the second aspect, the granular raw material 13 may be additionally cooled simultaneously with the coating of the surface of the granular raw material 13 with the powder 15 and/or after the coating of the surface of the granular raw material with the powder. The performance of the cooling can further secure the adhesion of the powder 15 to the granular raw material 13. The cooling may be, for example, natural cooling, or may be forced cooling.

When the natural cooling is performed, the solid particle 1 in which the surface of the granular raw material 13 is coated with the powder 15 only needs to be left at rest under room temperature.

When the forced cooling is performed, the following only needs to be performed: a gas is blown onto the solid particle 1; the solid particle 1 is left at rest in a refrigerator; or the solid particle 1 is brought into contact with a refrigerant.

The granular raw material 13 softens at the time point when the surface of the granular raw material 13 is coated with the powder 15. The granular raw material 13 whose surface has been coated with the powder 15 is solidified at any subsequent stage. The term “solidification” means that cooling makes the hardness of the granular raw material 13 equal to the hardness of the raw material composition before the fluidity is imparted thereto.

A portion free of the powder 15 in which the granular raw material 13 is solidified is the core portion of the solid particle 1. The granular raw material 13 is formed by granulating the raw material composition, and is in a state before its solidification. In addition, a portion where the raw material composition incorporates the powder 15 in the inside of the granular raw material 13 near its surface is the shell portion of the solid particle 1.

After the coating of the surface of the granular raw material with the powder, the powder 15 that does not stick to the surface of the solid particle 1 may be removed prior to the cooling of the solid particle 1, after the cooling, or simultaneously with the cooling. The term “powder 15 that does not stick to the surface of the solid particle 1” refers to the powder 15, which adheres to the surface to such a weak extent as to fall from the solid particle 1 owing to an external force such as vibration to be applied at the time of, for example, the conveyance of the solid particle 1, or which does not adhere to any portion derived from the raw material composition at all.

A sieve having such an aperture as not to pass the solid particle 1 therethrough may be used in the removal of the powder. In addition, as illustrated in FIG. 3, the solid particle 1 may be separated from the powder 15 and the satellite particle 3 through the sieve 24 after having been conveyed on the trough 21. The powder 15 may be reused by separately subjecting a mixture of the powder 15 and the satellite particle 3 to separation through a sieve.

### [Solid Particle]

The solid particle to be produced by the production method according to at least one embodiment of the present invention (hereinafter also referred to as “solid particle according to at least one embodiment of the present invention”) is a solid particle having a core-shell structure including a core portion formed of a granular solid raw material composition and a shell portion that coats at least part of the surface of the core portion. The shell portion is formed of a layer of the raw material composition having incorporated thereinto the powder. The powder may be a multilayer or a single layer, and a gap may be present between the particles for forming the powder.

In the solid particle according to at least one embodiment of the present invention, the strength of the inside of the solid particle near its surface is improved by the adhesion of the powder and its incorporation into the raw material composition. Accordingly, it may be possible to suppress, for example, the fracture, deformation, crushing, or collapse of the solid particle at the time of its transportation due to contact between the particles or between the particle and a container. Thus, the exposure of the core portion is suppressed, and adhesion between the solid particles can be suppressed. Accordingly, the solid particle may be excellent in transportation resistance. Further, a reduction in sense of use can be suppressed because the powder is present near the surface.

The term “solid particle” refers to a particle that is a solid at room temperature (25° C.), and has such a property as to soften or melt to obtain fluidity when heated to a temperature higher than room temperature, for example, 50° C. or more. To obtain such solid particle, as described later, for example, a raw material composition having a melting point of 50° C. or more only needs to be used.

### [Shape of Solid Particle]

Although the size of the solid particle is not particularly limited, the average projected area of the particles when the particles are mounted on a plane is preferably 0.5 mm² or more, more preferably 1 mm² or more, still more preferably 1.5 mm² or more from the viewpoints of, for example, case of use such as the case with which the particles are taken in a hand, the difficulty with which the particles roll, the case with which the particles are crushed, a design property, and case of production. In addition, the average projected area of the solid particles is preferably 320 mm² or less, more preferably 80 mm² or less, still more preferably 20 mm² or less in terms of, for example, case of use such as the case with which the particles are taken in a hand, the case with which the particles are crushed, and a design property. In particular, the average projected area of the solid particles is preferably 0.5 mm² or more and 320 mm² or less, more preferably 1 mm² or more and 80 mm² or less, still more preferably 1.5 mm² or more and 20 mm² or less. The term “average projected area” means a number average determined as follows: 10 solid particles that have been randomly sampled are adopted as measurement objects; and under a state in which the solid particles are mounted in the most stable state on a horizontal plane, areas projected onto the horizontal plane with light from directly above are measured, and their number average is determined.

When the solid particle has a spherical shape or a substantially spherical shape, its diameter is preferably 0.5 mm or more, more preferably 1 mm or more, still more preferably 1.5 mm or more, and is preferably 20 mm or less, more preferably 10 mm or less, still more preferably 5 mm or less. The diameter of the solid particle having a spherical shape or a substantially spherical shape is preferably 0.5 mm or more and 20 mm or less, more preferably 1 mm or more and 10 mm or less, still more preferably 1.5 mm or more and 5 mm or less.

In addition, when the solid particle has a spheroidal shape or a substantially spheroidal shape, its diameter is a circle-equivalent diameter determined from the above-mentioned horizontal projection. The diameter is preferably 0.5 mm or more, more preferably 1 mm or more, still more preferably 1.5 mm or more, and is preferably 20 mm or less, more preferably 10 mm or less, still more preferably 5 mm or less. The diameter of the solid particle having a spheroidal shape or a substantially spheroidal shape is preferably 0.5 mm or more and 20 mm or less, more preferably 1 mm or more and 10 mm or less, still more preferably 1.5 mm or more and 5 mm or less.

Further, when the solid particle has a spheroidal shape or a substantially spheroidal shape, its height is a distance between a plane that is brought into contact with the solid particle, the plane being horizontal to the horizontal plane on which the solid particle is mounted, and being positioned at a place most distant therefrom, and the horizontal plane. The height is preferably 0.4 mm or more, more preferably 0.8 mm or more, still more preferably 1.2 mm or more, and is preferably 16 mm or less, more preferably 8 mm or less, still more preferably 4 mm or less. The height of the solid particle having a spheroidal shape or a substantially spheroidal shape is preferably 0.4 mm or more and 16 mm or less, more preferably 0.8 mm or more and 8 mm or less, still more preferably 1.2 mm or more and 4 mm or less.

The average mass of the solid particles per particle is preferably 1 mg or more, more preferably 5 mg or more, still more preferably 10 mg or more. In addition, the average mass of the solid particles per particle is preferably 10,000 mg or less, more preferably 5,000 mg or less, still more preferably 1,000 mg or less. The average mass of the solid particles is preferably 1 mg or more and 10,000 mg or less, more preferably 5 mg or more and 5,000 mg or less, still more preferably 10 mg or more and 1,000 mg or less. The term “average mass” means the number average of the masses of 10 solid particles that have been randomly sampled.

The thickness of the shell portion of the solid particle, which depends the properties of the powder and production conditions for the solid particle, is preferably 80 μm or more, more preferably 100 μm or more, still more preferably 110 μm or more, still further more preferably 120 μm or more, still further more preferably 130 μm or more, still further more preferably 150 μm or more from the viewpoint of improving the adhesion resistance and transportation resistance thereof. In particular, the thickness of the shell portion is preferably 110 μm or more because the adhesion resistance and transportation resistance of the solid particle are further improved. In addition, the thickness is preferably 300 μm or less, more preferably 250 μm or less, still more preferably 200 μm or less from the viewpoint of suppressing a reduction in sense of use of the solid particle.

The thickness of the shell portion includes thicknesses in the following cases: a case in which the powder having a diameter smaller than the above-mentioned thickness forms a multilayer; and a case in which the powder having a diameter equal to the above-mentioned thickness forms a single layer.

The content of the powder in the solid particle is preferably 5.0 mass % or more, more preferably 7.0 mass % or more, still more preferably 7.5 mass % or more, still further more preferably 8.0 mass % or more in terms of ratio with respect to the mass of the solid particle from the viewpoint of improving the adhesion resistance and transportation resistance of the solid particle. In addition, the content is preferably 30 mass % or less, more preferably 20 mass % or less, still more preferably 15 mass % or less, still further more preferably 13 mass % or less, still further more preferably 12 mass % or less from the viewpoint of eliminating an influence on the sense of use thereof.

The average strength of the solid particles per particle is preferably 0.14 N or more, more preferably 0.16 N or more from the viewpoint of the transportation resistance. In addition, the average strength of the solid particles per particle is preferably 3 N or less, more preferably 1 N or less from the viewpoint of the sense of use. The average strength of the solid particles is preferably 0.14 N or more and 3 N or less, more preferably 0.16 N or more and 1 N or less. The term “average strength” means the number average of the strengths of 10 solid particles that have been randomly sampled. The strength of the solid particle is measured by a method described in Examples.

### [Raw Material for Solid Particle]

### [Raw Material Composition]

The raw material composition is a solid at room temperature (25° C.), and has a melting point of preferably 50° C. or more, more preferably 55° C. or more, still more preferably 60° C. or more. When the raw material composition has a melting point equal to or more than those temperatures, the sense of use of the solid particle at the time of its use as a cosmetic can be improved because the granular raw material obtained by granulating the raw material composition serves as the core portion of the solid particle. In addition, the melting point of the raw material composition is preferably 150° C. or less, more preferably 120° C. or less, still more preferably 110° C. or less from the viewpoint of case of production. Also when the raw material composition has a melting point equal to or less than those temperatures, the sense of use of the solid particle can be improved. In particular, the melting point of the raw material composition is preferably 50° C. or more and 150° C. or less, more preferably 55° C. or more and 120° C. or less, still more preferably 60° C. or more and 110° C. or less.

The raw material composition typically contains a plurality of substances. In this case, the melting point of the raw material composition is measured by any one of the first method, second method, and third method of the general test methods of the Japanese Standards of Quasi-drug Ingredients. Which one of the methods is adopted is selected mainly by the melting point of the raw material composition. When the melting point is as high as more than 75° C., the first method may be used, when the melting point is 50° C. or more and 75° C. or less, the second method may be used, and when the melting point is less than 50° C., the third method may be used.

The raw material composition is preferably a cosmetic raw material composition from the viewpoint that the solid particle according to at least one embodiment of the present invention is preferably used as a cosmetic.

The raw material composition preferably has a continuous phase formed of one or two or more kinds of oily components. In some cases, the raw material composition contains a powder component such as a pigment dispersed in the continuous phase.

Examples of the oily component include a hydrocarbon oil, an ester oil, an ether oil, a fatty acid, an alcohol, a silicone oil, and a fluorine oil. The number of carbons in the oily component is preferably 6 or more, more preferably 10 or more, and is preferably 50 or less, more preferably 30 or less. Specific examples of the oily component include: waxes including: mineral waxes, such as ozokerite and ceresin; petroleum waxes, such as a paraffin and a microcrystalline wax; synthetic hydrocarbons, such as a Fischer-Tropsch wax, a polyethylene wax, and a synthetic wax; plant waxes, such as a carnauba wax, a candelilla wax, a rice wax, a sunflower wax, a hydrogenated jojoba oil, and a Japanese wax; animal waxes, such as a beeswax and a whale wax; and synthetic waxes, such as a silicone wax, a synthetic beeswax, and a synthetic Japanese wax; oily gelling agents, such as dextrin palmitate, a sucrose fatty acid ester, inulin stearate, 12-hydroxystearic acid, dibutyl lauroyl glutamide, dibutyl ethylhexanoyl glutamide, and a polyamide resin; paste oils, such as vaseline, a vinyl leather wax, dipentaerythrityl hexa (behenate/benzoate/ethylhexanoate), cholesteryl hydroxystearate, dipentaerythrityl tetra(hydroxystearate/isostearate), a hydrogenated palm oil, dipentaerythrityl hexahydroxystearate, glyceryl tri (caprylate/caprate/myristate/stearate), dipentaerythrityl hexa (hydroxystearate/stearate/rosinate), phytosteryl oleate, glyceryl (ethylhexanoate/stearate/adipate), di(octyldodecyl/phytosteryl/behenyl) lauroyl glutamate, (phytosteryl/isostearyl/cetyl/stearyl/behenyl) dimer dilinoleate, dimer dilinoleyl bis(behenyl/isostearyl/phytosteryl) dimer dilinoleate, hard lanolin, reduced lanolin, and bis-diglyceryl polyacyladipate-2; linear or branched hydrocarbon oils, such as a liquid paraffin, a light liquid isoparaffin, a heavy liquid isoparaffin, a mineral oil, squalane, an α-olefin oligomer, polyisobutylene, polybutene, hydrogenated polyisobutene, and hydrogenated polydecene; ester oils, such as isononyl isononanoate, isodecyl isononanoate, isotridecyl isononanoate, tricyclodecanemethyl isononanoate, ethyl isostearate, isobutyl isostearate, isopropyl isostearate, 2-hexyldecyl isostearate, di-2-ethylhexyl succinate, bis-ethoxydiglycol succinate, hexyl laurate, propanediol di(caprylate/caprate), neopentyl glycol diisononanoate, neopentyl glycol dicaprate, glyceryl diisostearate, polyglyceryl diisostearate, propanediol diisostearate, trimethylolpropane triisostearate, glyceryl triisostearate, diglyceryl triisostearate, diglyceryl tetraisostearate, diisostearyl malate, octyldodecyl malate, a glycerin fatty acid ester, a jojoba oil, di(phytosteryl/octyldodecyl) lauroyl glutamate, octyldodecyl myristate, isopropyl myristate, 2-ethylhexyl palmitate, isopropyl palmitate, cetyl 2-ethylhexanoate, trimethylolpropane tri-2-ethylhexanoate, glyceryl tri-2-ethylhexanoate, octyldodecyl myristate, 2-hexyldecyl myristate, 2-hexyldecyl 2-ethylhexanoate, neopentyl glycol di-2-ethylhexanoate, ethylhexyl hydroxystearate, glyceryl tri (caprylate/caprate), glyceryl trioctanoate, neopentyl glycol dioctanoate, tridecyl trimellitate, dipentaerythrityl tetraisostearate, pentaerythrityl tetraisostearate, octyl methoxycinnamate, 2-ethylhexyl paramethoxycinnamate, diisopropyl dimerate, and propylene carbonate; higher alcohols, such as lauryl alcohol, oleyl alcohol, isostearyl alcohol, behenyl alcohol, and octyldodecanol; silicone oils, such as dimethylpolysiloxane, dimethylcyclopolysiloxane, methylphenylpolysiloxane, trimethylpentaphenyltrisiloxane, methylhydrogenpolysiloxane, higher alcohol-modified organopolysiloxane, and bisalkyl (C16-18) glycerin undecyl dimethicone; fluorine oils, such as a fluoropolyether, a perfluoroalkyl ether silicone, and a fluorine-modified silicone; and tocopherol, dipropylene glycol, and phenoxyethanol. Those oily components may be used alone or in combination thereof.

The raw material composition contains preferably 40 mass % or more, more preferably 50 mass % or more, still more preferably 60 mass % or more, still more preferably 70 mass % or more, still more preferably 75 mass % or more of the oily component in terms of the sense of use of the solid particle. From the same viewpoint, the raw material composition contains preferably 100 mass % or less, more preferably 99 mass % or less, still more preferably 98 mass % or less, still more preferably 97 mass % or less, still more preferably 95 mass % or less of the oily component. In particular, the raw material composition contains preferably 40 mass % or more and 100 mass % or less, more preferably 50 mass % or more and 99 mass % or less, still more preferably 60 mass % or more and 98 mass % or less, still more preferably 70 mass % or more and 97 mass % or less, still more preferably 75 mass % or more and 95 mass % or less of the oily component.

The raw material composition preferably contains an oily component that is in a solid state at 20° C. (has a melting point of more than 20° C.) (e.g., any one of the above-mentioned waxes) from the viewpoint of improving the shape-retaining property of the solid particle. The raw material composition contains preferably 5 mass % or more, more preferably 7 mass % or more of the oily component that is in a solid state at 20° C. from the viewpoint of the shape-retaining property of the solid particle. In addition, the raw material composition contains preferably 30 mass % or less, more preferably 10 mass % or less of the oily component that is in a solid state at 20° C. from the viewpoint of the sense of use of the solid particle. In particular, the raw material composition contains preferably 5 mass % or more and 30 mass % or less, more preferably 7 mass % or more and 10 mass % or less of the oily component that is in a solid state at 20° C. In addition, the raw material composition preferably contains an oily component that is in a liquid state at 20° C. (has a melting point of 20° C. or less) from the viewpoint of improving the sense of use of the solid particle. From this viewpoint, the raw material composition contains preferably 18 mass % or more, more preferably 25 mass % or more, still more preferably 35 mass % or more of the oily component that is in a liquid state at 20° C. From the same viewpoint, the raw material composition contains preferably 95 mass % or less, more preferably 93 mass % or less, still more preferably 90 mass % or less of the oily component that is in a liquid state at 20° C. In particular, the raw material composition contains preferably 18 mass % or more and 95 mass % or less, more preferably 25 mass % or more and 93 mass % or less, still more preferably 35 mass % or more and 90 mass % or less of the oily component that is in a liquid state at 20° C.

Whether or not the oily component is in a liquid state at 20° C. may be judged by contents described in the section “Physical State” of the safety data sheet (SDS) of each component. When the oily component that is in a liquid state at 20° C. is incorporated in the above-mentioned ranges into the raw material composition, the oily component is present on the surface of the core portion of the solid particle after its production, and the core portion is liable to be bonded to any other thing owing to the fact. In contrast, in the solid particle produced by the production method according to at least one embodiment of the present invention, the surface of the core portion is coated with the shell portion. Accordingly, unintended bonding between the solid particles or between the solid particle and any other thing is suppressed, and hence the solid particle has adhesion resistance.

For example, various components that have heretofore been used in cosmetics may each be used as the powder component in the raw material composition without any particular limitation. The powder component may be inorganic powder, or may be organic powder. The inorganic powder and the organic powder may be used in combination. The shape of a particle for forming the powder component is not particularly limited, and may be, for example, a spherical shape, a polyhedral shape, a flake shape, a spindle shape, a fibrous shape, an indefinite shape, or a combination thereof.

Pigments, such as a coloring pigment, a luster pigment, and an extender pigment, may each be used as the powder component in the raw material composition, and an inorganic powder pigment is preferred.

Examples of the coloring pigment include: metal oxides, such as titanium oxide, zinc oxide, yellow iron oxide, red iron oxide, black iron oxide, iron blue, ultramarine blue, chromium oxide, and chromium hydroxide; metal complexes, such as manganese violet and cobalt titanate; inorganic pigments such as carbon black; synthetic organic pigments, such as Red No. 3, Red No. 104, Red No. 106, Red No. 201, Red No. 202, Red No. 204, Red No. 205, Red No. 220, Red No. 226, Red No. 227, Red No. 228, Red No. 230, Red No. 401, Red No. 405, Red No. 505, Orange No. 203, Orange No. 204, Orange No. 205, Yellow No. 4, Yellow No. 5, Yellow No. 401, Blue No. 1, and Blue No. 404; and natural organic colors, such as β-carotene, caramel, and a paprika color.

Examples of the luster pigment include: a pigment obtained by coating the surface of sheet-shaped powder of mica, synthetic fluorophlogopite, glass, silica, alumina, or talc with a colorant including titanium oxide, iron oxide, silicon oxide, iron blue, chromium oxide, tin oxide, chromium hydroxide, gold, silver, carmine, or an organic pigment, such as Red No. 202 or Yellow No. 4; and a pigment obtained by cutting a raw material film of polyethylene terephthalate/polymethyl methacrylate laminated powder, polyethylene terephthalate/aluminum deposited powder, or polyethylene terephthalate/gold deposition-laminated powder into any appropriate shape.

Examples of the extender pigment include inorganic powders including silica, mica, synthetic fluorophlogopite, glass powder, barium sulfate, kaolin, bentonite, hectorite, zeolite, bismuth oxychloride, zirconium oxide, magnesium oxide, aluminum oxide, calcium sulfate, barium sulfate, magnesium sulfate, calcium carbonate, magnesium carbonate, and talc.

Further examples thereof include organic powders including nylon, polyethylene, a silicone elastomer such as a (vinyl dimethicone/methicone silsesquioxane) crosspolymer, polymethyl methacrylate, lauroyl lysine, silk powder, cellulose powder, a dispersant such as a polyvalent metal salt of a long-chain fatty acid, and various kinds of wax powder.

The ratio of the powder component in the raw material composition is preferably 0.01 mass % or more, more preferably 0.1 mass % or more, still more preferably 1 mass % or more, still more preferably 3 mass % or more, still more preferably 5 mass % or more. In addition, the ratio of the powder component in the raw material composition is preferably 60 mass % or less, more preferably 50 mass % or less, still more preferably 45 mass % or less, still more preferably 30 mass % or less, still more preferably 20 mass % or less. In particular, the ratio of the powder component in the raw material composition is preferably 0.01 mass % or more and 60 mass % or less, more preferably 0.1 mass % or more and 50 mass % or less, still more preferably 1 mass % or more and 45 mass % or less, still more preferably 3 mass % or more and 30 mass % or less, still more preferably 5 mass % or more and 20 mass % or less.

The hardness of the core portion is one factor that affects, for example, the sense of use of the solid particle. From this viewpoint, the hardness of the raw material composition is preferably 500 g or less, more preferably 350 g or less, still more preferably 250 g or less, still further more preferably 150 g or less. Meanwhile, unintended bonding between the raw material composition and any other thing can be suppressed by setting the hardness of the raw material composition to preferably 0.5 g or more, more preferably 5 g or more, still more preferably 15 g or more. From those viewpoints, the hardness of the raw material composition is preferably 0.5 g or more and 500 g or less, more preferably 5 g or more and 350 g or less, still more preferably 15 g or more and 250 g or less, still further more preferably 15 g or more and 150 g or less. The hardness of the raw material composition is measured by a method described in Examples.

### [Powder]

The ratio (powder adhesion ratio) of the powder to be used in the coating of the surface of the granular raw material with the powder to the mass of the solid particle is preferably 4.0 mass % or more, more preferably 4.5 mass % or more, still more preferably 5.0 mass % or more, still further more preferably 6.0 mass % or more from the viewpoints of the adhesion resistance and transportation resistance of the solid particle. In addition, the ratio is preferably 30 mass % or less, more preferably 20 mass % or less, still more preferably 15 mass % or less, still further more preferably 13 mass % or less, still further more preferably 12 mass % or less from the viewpoint of eliminating an influence on the sense of use thereof.

The same powder as that generally used in a cosmetic may be used as the powder without any particular limitation. The powder may be inorganic powder, and the inorganic powder may be a pigment. Alternatively, the powder may be organic powder. Although the inorganic powder and the organic powder may be used in combination, the inorganic powder is preferably incorporated and silica is more preferred.

Specifically, the same component as the powder component in the raw material composition described above may be used.

Powder having such a size that the adhesion of the solid particle serving as the object of the production method according to at least one embodiment of the present invention to any other thing in its production process is effectively prevented is suitably used as the powder. For example, when the sizes of particles for forming the powder are represented by a volume cumulative particle diameter D₅₀ at a cumulative volume of 50 vol % measured by a laser diffraction/scattering particle size distribution measurement method, the D₅₀ is preferably 0.01 μm or more, more preferably 0.1 μm or more, still more preferably 1 μm or more, still more preferably 5 μm or more, still more preferably 10 μm or more. The use of the powder having such size facilitates the performance of the operation of removing the powder. In addition, the D₅₀ is preferably 500 μm or less, more preferably 300 μm or less, still more preferably 160 μm or less, still more preferably 100 μm or less, still more preferably 30 μm or less. The use of the powder having such size can facilitate the adhesion of the powder to the surface of the granular raw material. In particular, the D₅₀ is preferably 0.01 μm or more and 500 μm or less, more preferably 0.1 μm or more and 300 μm or less, still more preferably 1 μm or more and 160 μm or less, still more preferably 5 μm or more and 100 μm or less, still more preferably 10 μm or more and 30 μm or less.

The oil absorption of the powder is preferably 5 mL/100 g or more, more preferably 15 mL/100 g or more, still more preferably 20 mL/100 g or more from the viewpoint of effectively preventing the adhesion of the solid particle to any other thing in its production process. In addition, the oil absorption of the powder is preferably 500 mL/100 g or less, more preferably 400 mL/100 g or less, still more preferably 350 mL/100 g or less from the viewpoint of preventing the powder from excessively absorbing the oily component in the granular raw material. The oil absorption is measured in conformity with JIS K 5101-13-1:2004.

The amount of the powder in the solid particle may be determined and measured by various methods. For example, when the powder is inorganic matter, its amount may be determined by burning organic matter in the solid particle and measuring its remaining ratio with a thermogravimetric differential thermal analyzer as described below. The amount of the powder that is inorganic matter is measured by a method described in Examples.

When the powder is organic matter, the amount of the powder adhering to the core portion may be determined as follows: a product obtained by dissolving all the components of the solid particle in a solvent is used as a sample; and the sample is subjected to measurement, such as ¹H-NMR or laser desorption/ionization mass spectrometry (LDI-MS).

The solid particle produced by the production method according to at least one embodiment of the present invention is preferably used as a cosmetic. The solid particle may be used in, for example, a makeup method including crushing the particle and applying the crushed particle to a human body for cosmetic purposes. Specifically, the following may be performed: the solid particle is mounted on a cosmetic palette, and is crushed with a makeup brush, followed by its application to a lip with the makeup brush like a lipstick. Alternatively, the following may be performed: the solid particle is crushed on the back of a hand, and is applied to a check with a finger like a blusher or a concealer. Alternatively, the following may be performed: the solid particle is crushed on the back of a hand, and is applied to a finger or the back of the hand with the finger like a hand cream. Further, the solid particle may be used like an oil cleansing by being crushed with a hand. Further, the solid particle may be used like a treatment or a hair wax by being crushed with a hand or a tool and applied to hair.

The present invention has been described above on the basis of its exemplary embodiments. However, the present invention is not limited to the embodiments.

According to the production method according to at least one embodiment of the present invention, a solid particle to which no fine particle adheres can be provided.

### EXAMPLES

The present invention is described in more detail below by way of Examples. However, the scope of the present invention is not limited to these Examples.

### [Preparation of Raw Material Composition]

The composition of a raw material composition for a lipstick is shown in Table 1 below, and the composition of a raw material composition for an eye shadow is shown in Table 2 below. In each of the raw material compositions, base raw materials were heated and dissolved at 110° C. for 30 minutes, and were uniformly mixed with a disper. Next, a coloring pigment, a luster pigment, and an extender pigment, or the coloring pigment, the luster pigment, and a dispersant were added to the base raw materials, and the materials were further uniformly mixed for 15 minutes, followed by degassing. After that, the mixture was naturally cooled to be solidified. Thus, the raw material compositions were prepared. The melting points of the prepared raw material compositions were measured in accordance with the general test methods of the Japanese Standards of Quasi-drug Ingredients. As a result, the raw material composition for a lipstick had a melting point of 68° C., and the raw material composition for an eye shadow had a melting point of 81° C.

The raw material composition for a lipstick prepared in the foregoing was heated and dissolved at 115° C., and was filled into a resin-made ointment jar (having a diameter of 30 mm and a height of 14 mm) up to a height of 10 mm. After that, the composition was cooled at 20° C. for 2 hours to be solidified, and was left at rest at 30° C. for 6 hours or more. After that, the hardness of the raw material composition for a lipstick was measured with a rheometer manufactured by Rheotech by reading the maximum of a load when a jig having a diameter of 2 mm was caused to penetrate into the composition to a depth of 2 mm at a table speed of 2 mm/s. As a result, the hardness was 35 g.

The hardness of the raw material composition for an eye shadow prepared in the foregoing was measured under the same conditions. As a result, the hardness was 41 g.

TABLE 1

| | | Ratio |
|---|---|---|
| | | (mass %) |
| Base | Paraffin | 4.0 |
| | Polyethylene wax | 2.0 |
| | Microcrystalline wax | 2.0 |
| | (Multi-branched isostearic acid) | 30.0 |
| | dipentaerythrityl tetraisostearate | |
| | Hydrogenated polyisobutene | 10.0 |
| | Di(phytosteryl/octyldodecyl) lauroyl glutamate | 10.0 |
| | Octyldodecanol | 15.0 |
| | Bisalkyl (C16-18) glycerin undecyl dimethicone | 6.0 |
| | Glyceryl tri(caprylate/caprate) | 10.2 |
| | Tocopherol | 0.1 |
| | Dipropylene glycol | 0.1 |
| Coloring | Red No. 202 | 0.3 |
| pigment | Yellow No. 4 Aluminum Lake | 0.2 |
| | Yellow iron oxide | 0.5 |
| | Red iron oxide | 0.1 |
| | Titanium oxide | 1.0 |
| Luster | Mica titanium | 2.0 |
| pigment | Titanium oxide-coated glass flake | 1.5 |
| Extender | Mica | 5.0 |
| pigment | | |

| | |
|---|---|
| Total | 100.0 |

TABLE 2

| | | Ratio |
|---|---|---|
| | | (mass %) |
| Base | Paraffin | 4.0 |
| | Synthetic wax | 3.0 |
| | Microcrystalline wax | 2.0 |
| | (Multi-branched isostearic acid) | 25.0 |
| | dipentaerythrityl tetraisostearate | |
| | Polyglyceryl-2 triisostearate | 5.0 |
| | Polyglyceryl-2 diisostearate | 5.0 |
| | Isotridecyl isononanoate | 15.0 |
| | Glyceryl tri(caprylate/caprate) | 20.45 |
| Coloring | Red No. 202 | 0.05 |
| pigment | | |
| Luster | Titanium oxide-coated glass flake | 20.0 |
| pigment | | |
| Dispersant | Zinc stearate | 0.5 |

| | |
|---|---|
| Total | 100.0 |

As shown in Table 1, the amount of oily components in the raw material composition for a lipstick was the amount of components shown as bases, that is, 89.4 mass %, and the amount of powder components therein was the total amount of the coloring pigments, the luster pigments, and the extender pigment, that is, 10.6 mass %. In addition, the amount of oily components each of which had a melting point of more than 20° C. (was a solid at 20° C.) in the raw material composition for a lipstick was the total amount of the paraffin, the polyethylene wax, and the microcrystalline wax, that is, 8 mass %, and the amount of oily components each of which had a melting point of 20° C. or less (was a liquid at 20° C. or less) therein was the total amount of the other components, that is, 81.4 mass %.

As shown in Table 2, the amount of oily components in the raw material composition for an eye shadow was the amount of components shown as bases, that is, 79.45 mass %, and the amount of powder components therein was the total amount of the coloring pigment, the luster pigment, and the dispersant, that is, 20.55 mass %. In addition, the amount of oily components each of which had a melting point of more than 20° C. (was a solid at 20° C.) in the raw material composition for an eye shadow was the total amount of the paraffin, the synthetic wax, and the microcrystalline wax, that is, 9.0 mass %, and the amount of oily components each of which had a melting point of 20° C. or less (was a liquid at 20° C. or less) therein was the total amount of the other components, that is, 70.45 mass %.

### [Measurement of Maximum Length of Elongation of Raw Material Composition at Time of its Granulation]

The raw material composition for a lipstick and the raw material composition for an eye shadow prepared in accordance with Table 1 and Table 2 above were each measured for the maximum length of the elongation of the raw material composition dripping from the nozzle 12 at the time of its granulation through ejection from the nozzle 12 at a dropping temperature and a dropping flow rate shown in Table 3. The maximum length of the elongation was observed with a high-speed camera (manufactured by Kron Technologies Inc., Chronos 1.4, frame rate: 500 fps), and was defined as a distance from the tip of the nozzle 12 to the lower end of a base particle in a frame immediately before each of the elongated raw material compositions was cut at any point. The maximum length of the elongation was measured three times for each composition, and an average of the measured values was 13.8 mm for the raw material composition for a lipstick and 17.1 mm for the raw material composition for an eye shadow.

### Example 1-1

### Production of Granular Raw Material

The plurality of granular raw materials 13 were formed from the raw material composition for a lipstick with the apparatus illustrated in FIG. 3. That is, the raw material composition 10 for a lipstick having imparted thereto fluidity through the melting of the raw material composition by heating to 90° C. was delivered with the pump 11 at 5.2 mL/min, and was ejected from the tip of the nozzle 12 having an inner diameter of 2.0 mm and an outer diameter of 3.2 mm at a dropping temperature and by a dropping distance, the temperature and the distance being shown in Table 3. Thus, the granular raw materials 13 serving as droplets were formed. The term “dropping distance” refers to a distance between the tip of the nozzle 12 and the outermost surface of the layer of the powder 15. The term “dropping temperature” refers to a temperature of the raw material composition 10 at the time of its ejection from the nozzle 12.

Dropping of Granular Raw Material into Powder and Coating thereof with Powder

The granular raw materials 13 formed in the foregoing were dropped at a distance 5.5 times as large as the maximum length of the elongation of the raw material composition at the time of its granulation onto the trough 21 to which the powder 15 at a temperature of 25° C. having applied thereto vibration having an amplitude of 0.663 mm and a frequency of 54.0 Hz by the vibration feeder 23 (SMALL ELECTROMAGNETIC FEEDER CF-2 manufactured by Sinfonia Technology Co., Ltd.) including the vibration device 22 and the trough 21 was supplied and mounted so as to have a thickness of 10 mm, and the powder 15 was caused to adhere to the surface of each of the granular raw materials 13 by bringing the granular raw materials 13 and the powder 15 into contact with each other.

Spherical silica having an average particle diameter D₅₀ of 15 μm and an oil absorption of 150 mL/100 g was used as the powder 15. Thus, the target solid particles 1 for a lipstick in each of which the granular raw material 13 was coated with the powder 15 by the adhesion of the powder 15 to the surface of the granular raw material 13 were obtained.

The value of the amplitude is a value obtained by measuring an amplitude directly above the vibration device 22 on the upper surface of the trough 21 with a laser displacement meter (LK-G 5000 manufactured by Keyence Corporation). The measurement was performed under the following conditions: the measurement was performed in a diffusion-reflection mode at a sampling period of 200 μs (5 kHz) and a moving average of 4.

### (Powder Removal and Cooling)

After that, the powder 15 and the solid particles 1 were separated from each other with the sieve 24 having an aperture of 2,000 μm under room temperature (25° C.) so that the powder 15 that did not adhere to the solid particles 1 was removed. The solid particles 1 were naturally cooled on the sieve 24 for 1 minute or more, and were recovered.

### [Evaluation of Solid Particles 1]

Ten particles were randomly sampled from the resultant solid particles 1 (provided that a particle to which the fine particle 14 adhered was excluded), and were evaluated. As a result, the ten particles had an average mass of 20.8 mg, an average diameter of 4.1 mm, an average height of 2.8 mm and an average powder adhesion ratio of 9.8 mass %.

### <Powder Adhesion Ratio>

The amount (adhesion ratio) of the silica used as the powder 15 in the solid particles 1 was determined as described below.

Ten particles were randomly sampled from the resultant solid particles 1, and the temperature of each of the ten particles was increased from 25° C. to 600° C. at 10° C./min with a thermogravimetric differential thermal analyzer “TG-DTA EXSTAR 6200” manufactured by Hitachi High-Tech Science Corporation while air was supplied at 200 mL/min, followed by the measurement of the mass of the residue after the burning. A value calculated from the following equation by using the mass of the residue when only a raw material composition having the same mass as that of the raw material composition used in the production of the solid particles 1 was subjected to the same treatment was defined as a powder adhesion ratio. An average of the powder adhesion ratios of the ten solid particles 1 is defined as an average powder adhesion ratio.

(Powder adhesion ratio (mass %))=(mass % of residue of solid particles 1)−[(mass % of residue of raw material composition)/[100−(mass % of residue of raw material composition)]]×[100−(mass % of residue of solid particles 1)]

### <Fine Particle Adhesion Ratio>

150 particles were randomly sampled from the resultant solid particles 1, and whether or not the surfaces were each coated with the powder 15 was visually determined under a state in which the fine particles 14 adhered to the surfaces of the granular raw materials 13. Solid particles 1-1 without fine particle adhesion (the granular raw materials 13 and the fine particles 14 coalesced with each other, followed by coating with the powder 15) illustrated in FIG. 6, and solid particles 1-2 with fine particle adhesion (the particles in which the fine particles 14 adhered to the surfaces of the granular raw materials 13 were each coated with the powder 15) illustrated in FIG. 7 were selected, and a fine particle adhesion ratio (%) was calculated by the following equation. In the solid particles of Example 1-1, the fine particle adhesion ratio was 7.6%.

Fine particle adhesion ratio (%)=(number of solid particles 1-2/number of solid particles 1)×100

### Examples 1-2 and 1-3

The solid particles 1 for lipsticks were each produced in the same manner as in Example 1-1 except that the respective conditions of the production method were changed as shown in Table 3. Results obtained by evaluating the resultant solid particles 1 in the same manner as in Example 1-1 are shown in Table 3.

### Examples 1-4 to 1-9

The solid particles 1 for eye shadows were each produced in the same manner as in Example 1-1 except that: the raw material composition was changed to the raw material composition for an eye shadow; and the respective conditions of the production method were changed as shown in Table 3. Results obtained by evaluating the resultant solid particles 1 in the same manner as in Example 1-1 are shown in Table 3.

### Comparative Examples 1-1 and 1-2

The solid particles 1 for lipsticks were each produced in the same manner as in Example 1-1 except that the respective conditions of the production method were changed as shown in Table 3. Results obtained by evaluating the resultant solid particles 1 in the same manner as in Example 1-1 are shown in Table 3. Under the production conditions of Comparative Example 1-2, the granular raw material was not able to be cut from the liquid column. Thus, the granular raw materials 13 were not able to be produced, and the solid particles 1 were not able to be produced.

TABLE 3

| | | | Example | | | | | | | | | Comparative Example | |
|---|---|---|---|---|---|---|---|---|---|---|---|---|---|
| | | | 1-1 | 1-2 | 1-3 | 1-4 | 1-5 | 1-6 | 1-7 | 1-8 | 1-9 | 1-1 | 1-2 |
| Granular | Raw material composition | | Lip- | Lip- | Lip- | Eye | Eye | Eye | Eye | Eye | Eye | Lip- | Lip- |
| raw material | | | stick | stick | stick | shadow | shadow | shadow | shadow | shadow | shadow | stick | stick |
| | Melting point | [° C.] | 68 | 68 | 68 | 81 | 81 | 81 | 81 | 81 | 81 | 68 | 68 |
| Powder | Kind | | Silica | Silica | Silica | Silica | Silica | Silica | Silica | Silica | Silica | Silica | Silica |
| | Average particle | [μm] | 15 | 15 | 15 | 15 | 15 | 15 | 15 | 15 | 15 | 15 | 15 |
| | diameter | | | | | | | | | | | | |
| Production | Dropping temperature | [° C.] | 87 | 87 | 87 | 86 | 86 | 86 | 86 | 86 | 86 | 87 | 87 |
| condition | Dropping flow rate | [mL/ | 5.2 | 5.2 | 5.2 | 4.0 | 4.0 | 4.0 | 4.0 | 4.0 | 4.0 | 5.2 | 5.2 |
| | | min] | | | | | | | | | | | |
| | Dropping distance | [mm] | 76 | 41 | 6 | 13 | 38 | 60 | 80 | 100 | 128 | 128 | 0 |
| | Elongation distance *1 | [mm] | 13.8 | 13.8 | 13.8 | 17.1 | 17.1 | 17.1 | 17.1 | 17.1 | 17.1 | 13.8 | 13.8 |
| | Ratio *2 | [—] | 5.5 | 3.0 | 0.4 | 0.8 | 2.2 | 3.5 | 4.7 | 5.8 | 7.5 | 9.3 | 0.0 |
| Evaluation | Average mass | [mg] | 20.8 | 21.0 | 20.7 | 22.1 | 22.2 | 22.7 | 22.8 | 23.3 | 22.7 | 21. | Impossible |
| of solid | | | | | | | | | | | | | to evaluate |
| particle | Average diameter | [mm] | 4.1 | 4.1 | 4.2 | 4.4 | 4.4 | 4.6 | 4.5 | 4.6 | 4.9 | 4.3 | Impossible |
| | | | | | | | | | | | | | to evaluate |
| | Average height | [mm] | 2.8 | 2.6 | 2.9 | 3.1 | 3.0 | 3.1 | 2.9 | 2.8 | 3.1 | 2.9 | Impossible |
| | | | | | | | | | | | | | to evaluate |
| | Average powder | [mass | 9.8 | 10.0 | 9.8 | 6.4 | 6.9 | 7.2 | 8.3 | 7.3 | 6.9 | 9.2 | Impossible |
| | adhesion ratio | %] | | | | | | | | | | | to evaluate |
| | Fine particle | [number | 7.6 | 0.8 | 0.8 | 5.2 | 7.2 | 7.8 | 8.9 | 18.7 | 24.5 | 43.7 | Impossible |
| | adhesion ratio | %] | | | | | | | | | | | to evaluate |

*1: means the maximum length of the elongation of the raw material composition at the time of its granulation.
*2: means a ratio of the dropping distance to the elongation distance (dropping distance/elongation distance).

As shown in Table 3, according to the production methods of Examples 1-1 to 1-9, as a result of the coalescence of the granular raw materials 13 and the fine particles 14, followed by coating with the powder 15, the spherical solid particles 1 having satisfactory appearance were obtained in a high yield.

Meanwhile, according to the production method of Comparative Example 1-1, a large number of solid particles having unsatisfactory appearance in which the fine particles adhered to the surfaces of the solid particles were obtained.

As described above, according to the first aspect of the production method of the present invention, the adhesion of the fine particle to the solid particle is suppressed, and hence a solid particle having satisfactory appearance can be obtained in a high yield.

### Example 2-1

### Production of Granular Raw Material

The plurality of granular raw materials 13 were formed from the raw material composition 10 for a lipstick with the apparatus illustrated in FIG. 5. That is, the raw material composition 10 for a lipstick having imparted thereto fluidity through the melting of the raw material composition 10 for a lipstick by heating to 90° C. was delivered at 5.2 mL/min with the pump 11, and was ejected from the tip of the nozzle 12 having an inner diameter of 2.0 mm and an outer diameter of 3.2 mm. Thus, the granular raw materials 13 serving as droplets were formed. A distance between the tip of the nozzle 12 and the outermost surface of the layer of the powder 15 was 128 mm, and the temperature of the raw material composition 10 for a lipstick at the time of the ejection from the nozzle 12 was 87° C.

Dropping of Granular Raw Material into Powder and Coating Thereof with Powder

The granular raw materials 13 formed in the foregoing were dropped into the powder 15 on the trough 21 to which the powder 15 at a temperature of 25° C. having applied thereto vibration having an amplitude of 0.663 mm and a frequency of 54.0 Hz by the vibration feeder 23 (SMALL ELECTROMAGNETIC FEEDER CF-2 manufactured by Sinfonia Technology Co., Ltd.) including the vibration device 22 and the trough 21 was supplied and mounted so as to have a thickness of 10 mm, and the powder 15 was caused to adhere to the surface of each of the granular raw materials 13 by bringing the granular raw materials 13 and the powder 15 into contact with each other.

During a time period required for the granular raw material to fall from the nozzle to the powder, the airflow 18 was applied to a position of 25 cm in a perpendicular direction from the falling track of the granular raw material with a local exhaust ventilation device (manufactured by Amano Corporation, PiE-60D) to change only the track of the fine particle 14 formed secondarily at the time of the formation of the granular raw material 13, to thereby shift the falling position of the fine particle 14 to the powder 15. The direction of the airflow was set to a direction perpendicular to the dropping track, and the airflow rate of the local exhaust ventilation device in the dropping track was 0.29 m/sec (anemometer: manufactured by KANOMAX, ANEMOMASTER Model 6115).

Spherical silica having an average particle diameter D₅₀ of 15 μm and an oil absorption of 150 mL/100 g was used as the powder 15. Thus, the target solid particles 1 in each of which the granular raw material 13 was coated with the powder 15 by the adhesion of the powder 15 to the surface of the granular raw material 13 were obtained.

The value of the amplitude is a value obtained by measuring an amplitude directly above the vibration device 22 on the upper surface of the trough 21 with a laser displacement meter (LK-G 5000 manufactured by Keyence Corporation). The measurement was performed under the following conditions: the measurement was performed in a diffusion-reflection mode at a sampling period of 200 μs (5 kHz) and a moving average of 4.

### (Evaluation of Granular Raw Material 13 and Fine Particle 14)

The state of the raw material composition 10 for a lipstick after its ejection from the nozzle 12 and before its dropping into the powder 15 on the trough 21 was photographed with a high-speed camera, and a moving image was cut out as a still image. The still image was binarized and subjected to image analysis. Thus, the sizes of the granular raw material 13 and the fine particle 14 formed secondarily were measured. The numbers of pixels of the diameters of the granular raw material 13 and the fine particle 14 were each extracted based on a scale arranged in a screen, and the diameters of the granular raw material 13 and the fine particle 14 were converted into millimeters. The granular raw material 13 and the fine particle 14 falling from the nozzle 12 were each a true sphere, and hence a projected area was calculated from a radius that was a half of each of the above-mentioned diameters. In addition, the volume of a sphere having the above-mentioned radius was determined, and the mass of each of the granular raw material 13 and the fine particle 14 was determined from the specific gravity of 0.972 g/cm³ of the raw material composition 10 for a lipstick at the dropping temperature. Ten particles each of the granular raw materials 13 and the fine particles 14 were analyzed. Averages of the values, and, for the fine particle 14, ratios with respect to the granular raw material 13 (ratios to the granular raw material) are shown in Table 4.

### (Powder Removal and Cooling)

After that, the powder 15 and the solid particles 1 were separated from each other with the sieve 24 having an aperture of 2,000 μm under room temperature (25° C.) so that the powder 15 that did not adhere to the solid particles 1 was removed. The solid particles 1 were naturally cooled on the sieve 24 for 1 minute or more, and were recovered.

### [Evaluation of Solid Particles 1]

Ten particles were randomly sampled from the resultant solid particles 1 (provided that a particle to which the fine particle 14 adhered was excluded), and were evaluated. As a result, the ten particles had an average diameter of 4.5 mm, an average height of 2.5 mm, an average mass of 22.5 mg, and an average powder adhesion ratio of 10.6 mass %. The average powder adhesion ratio was determined by the method described in Example 1-1.

### <Fine Particle Adhesion Ratio>

A fine particle adhesion ratio was calculated in the same manner as in Example 1-1. As a result, the fine particle adhesion ratio was 1.9% in the solid particles of Example 2-1.

### Comparative Example 2-1

The solid particles 1 were produced in the same manner as in Example 2-1 except that the airflow 18 was not applied. The solid particles 1 had an average diameter of 4.4 mm, an average height of 2.8 mm, an average mass of 21.0 mg, and an average powder adhesion ratio of 11.5 mass %.

A fine particle adhesion ratio was calculated in the same manner as in Example 2-1. As a result, the fine particle adhesion ratio was 22.0% in the solid particles of Comparative Example 2-1.

TABLE 4

| | | | Example | Comparative |
|---|---|---|---|---|
| | | | 2-1 | Example 2-1 |
| Granular | Average diameter | [mm] | 3.2 | ← |
| raw | Average projected area | [mm²] | 8.0 | ← |
| material | Average mass (mg) | [mg] | 16.7 | ← |
| Fine | Average diameter | [mm] | 0.9 | ← |
| particle | (Ratio to granular | — | 0.28 | ← |
| | raw material) | | | |
| | Average projected area | [mm²] | 0.64 | ← |
| | (Ratio to granular | — | 0.08 | ← |
| | raw material) | | | |
| | Average mass | [mg] | 0.37 | ← |
| | (Ratio to granular | — | 0.02 | ← |
| | raw material) | | | |
| Evaluation | Average mass | [mg] | 4.5 | 4.4 |
| of solid | Average diameter | [mm] | 2.5 | 2.8 |
| particle | Average height | [mm] | 22.5 | 21.0 |
| | Average powder | [mass %] | 10.6 | 11.5 |
| | adhesion ratio | | | |
| | Fine particle | [%] | 1.9 | 22.0 |
| | adhesion ratio | | | |

As shown in Table 4, in the production method of Example 2-1, the spherical solid particles 1 in each of which the powder 15 adhered to the surface of the granular raw material 13 and the fine particle 14 did not adhere thereto were obtained in a high yield (98.1%). Meanwhile, in the production method of Comparative Example 2-1, a large number of solid particles in each of which the fine particle 14 adhered to the granular raw material 13 were obtained.

As described above, according to the second aspect of the production method of the present invention, the adhesion of the fine particle to the solid particle is suppressed, and hence a solid particle having satisfactory appearance can be obtained in a high yield.

### REFERENCE SIGNS LIST

1 solid particle
2 fine particle-adhered solid particle
3 satellite particle
10 raw material composition having imparted thereto fluidity
11 pump
12 nozzle
13 granular raw material
14 fine particle
15 powder
16 container
17 stirrer
18 airflow
21 trough
22 vibration device
23 vibration feeder
24 sieve

1. A production method for a solid particle, comprising:
heating a raw material composition containing an oily component to impart fluidity to the raw material composition;
granulating the raw material composition having imparted thereto the fluidity through ejection to form a granular raw material; and
dropping the granular raw material into powder to coat a surface of the granular raw material with the powder,
wherein, at a time of the formation of the granular raw material, a maximum length of elongation of the raw material composition at a time of the granulation of the raw material composition through the ejection is measured in advance, and a length of a dropping distance of the granular raw material is set so that a ratio of the length of the dropping distance to the maximum length of the elongation at the time of the granulation is more than 0 and 9 or less.

2. The production method for a solid particle according to claim 1, wherein the powder contains silica.

3. The production method for a solid particle according to claim 1, further comprising cooling the granular raw material.

4. The production method for a solid particle according to claim 1, further comprising removing the powder that is free from adhering to the solid particle.

5. The production method for a solid particle according to claim 1, wherein the heating comprises heating the raw material composition to a melting point thereof or more.

6. The production method for a solid particle according to claim 1, wherein the powder has an average particle diameter D₅₀ of 0.01 μm or more and 500 μm or less.

7. The production method for a solid particle according to claim 1, wherein the solid particle to be obtained has an average mass per particle of 1 mg or more and 10,000 mg or less.

8. The production method for a solid particle according to claim 1, wherein the coating of the surface of the granular raw material with the powder is performed under a state in which vibration is applied to the powder.

9. The production method for a solid particle according to claim 8, wherein the vibration has an amplitude of 0.3 mm or more and a frequency of 30 Hz or more.

10. A production method for a solid particle, comprising:
heating a raw material composition containing an oily component to impart fluidity to the raw material composition;
granulating the raw material composition having imparted thereto the fluidity through ejection to form a granular raw material; and
dropping the granular raw material into powder to coat a surface of the granular raw material with the powder,
wherein, at a time of the coating of the surface of the granular raw material with the powder, an airflow is applied from a side surface of a dropping track of the granular raw material.

11. The production method for a solid particle according to claim 10, wherein the powder contains silica.

12. The production method for a solid particle according to claim 10, further comprising cooling the granular raw material.

13. The production method for a solid particle according to claim 10, further comprising removing the powder that is free from adhering to the solid particle.

14. The production method for a solid particle according to claim 10, wherein the heating comprises heating the raw material composition to a melting point thereof or more.

15. The production method for a solid particle according to claim 10, wherein the powder has an average particle diameter D₅₀ of 0.01 μm or more and 500 μm or less.

16. The production method for a solid particle according to claim 10, wherein the solid particles to be obtained have an average mass per particle of 1 mg or more and 10,000 mg or less.

17. The production method for a solid particle according to claim 10, wherein the coating of the surface of the granular raw material with the powder is performed under a state in which vibration is applied to the powder.

18. The production method for a solid particle according to claim 17, wherein the vibration has an amplitude of 0.3 mm or more and a frequency of 30 Hz or more.
